(19) United States
(12) Patent Application Publication
(10) Pub. No.: US 2024/0399131 A1
(43) Pub. Date: Dec. 5, 2024

(54) ASEPTIC COUPLING SYSTEM WITH TRANSLATING SEAL MECHANISM

(71) Applicant: Nordson Corporation, Westlake, OH (US)

(72) Inventors: Jack Wise, Erie, CO (US); Casey Tyler Hebert, Fort Collins, CO (US); Frank Lombardi, Longmont, CO (US)

(21) Appl. No.: 18/698,386

(22) PCT Filed: Oct. 5, 2022
(86) PCT No.: PCT/US2022/045741

§ 371 Date: Apr. 4, 2024

Related U.S. Application Data
(60) Provisional application No. 63/252,839, filed on Oct. 6, 2021.

(51) Int. Cl. A61M 39/10; A61M 39/14; A61M 39/18; A61M 39/20

(52) U.S. Cl. CPC A61M 39/1011; A61M 39/14; A61M 39/18; A61M 39/20

(57) ABSTRACT

An aseptic coupling system includes a coupler and a connector including a body defining a plug portion and a connector fluid path and an aseptic cap releasably secured to an end of the plug portion. The coupler includes a housing defining a socket portion for receiving the plug portion and a cavity adjacent to the socket portion, a seal translating element assembled with the housing, and an actuator element connected to the seal translating element and operable to move the seal translating element from a cap receiving closed position to a cap displacing open position. When the seal translating element is moved from the closed position to the open position, the cap is disengaged from the connector plug portion and a coupler fluid path, defined by the seal translating element, is moved from an offset portion of the coupler housing cavity into alignment with the connector fluid path.

### CROSS-REFERENCE TO RELATED APPLICATION

This application claims priority to and all benefit of U.S. Provisional Patent Application Ser. No. 63/252,839, filed Oct. 6, 2021 and entitled ASEPTIC COUPLING SYSTEM WITH TRANSLATING SEAL MECHANISM, the entire disclosure of which is incorporated herein by reference.

### BACKGROUND

Pharmaceutical, biotechnology, and chemical industries rely on a processing system that can transfer fluids in a pure or sterile manner. As such, these industries rely on aseptic connectors to prevent unwanted organisms or media from contaminating the system.

Current aseptic coupling arrangements on the market require multiple steps to connect and disconnect, have a large footprint, require metal or mechanisms in the fluid path that can damage/contaminate cells or media, require gendered connectors, provide for only single use connection, and/or have mechanisms which lend itself to contamination concerns.

### SUMMARY OF THE DISCLOSURE

In accordance with an embodiment of one or more of the inventions presented in this disclosure, an aseptic coupling system includes first and second connectors and a coupler. Each of the first and second connectors includes a body defining a plug portion and a connector fluid path and an aseptic cap releasably secured to an end of the plug portion by an attachment feature on the cap that releasably engages the plug portion for attachment thereto. The coupler includes a housing defining first and second socket portions for receiving the plug portions of the first and second connectors and a cavity disposed between the first and second socket portions, a seal translating element assembled with the housing and at least partially disposed within the cavity of the coupler housing, and an actuator element connected to the seal translating element and operable to move the seal translating element from a cap receiving closed position to a cap displacing open position. When the seal translating element is in the cap receiving closed position, the caps of the first and second connectors are receivable in the cavity of the coupler housing in alignment with the seal translating element. When the seal translating element is moved from the cap receiving closed position to the cap displacing open position, the caps are disengaged from the connector plug portions and a coupler fluid path, defined by the seal translating element, is moved from an offset portion of the coupler housing cavity into alignment with the connector fluid paths.

In accordance with another embodiment of one or more of the inventions presented in this disclosure, an aseptic coupling system includes a coupler and at least one connector. The at least one connector includes a body defining a plug portion and a connector fluid path, and an aseptic cap releasably secured to an end of the plug portion by an attachment feature on the cap that releasably engages the plug portion for attachment thereto. The coupler includes a housing defining at least one socket portion for receiving the plug portion of the at least one connector and a cavity adjacent to the at least one socket portion, a seal translating element assembled with the housing and at least partially disposed within the cavity of the coupler housing, and an actuator element connected to the seal translating element and operable to move the seal translating element between a cap receiving closed position and a cap displacing open position. When the seal translating element is in the cap receiving closed position, a recessed portion of the seal translating element is positioned to receive the cap of the at least one connector. When the seal translating element is moved from the cap receiving closed position to the cap displacing open position, the cap is disengaged from the connector plug portion and displaced to a first offset portion of the coupler housing cavity, and a coupler fluid path, defined by the seal translating element, is moved from a second offset portion of the coupler housing cavity into alignment with the connector fluid path. The coupler further comprises sealing arrangements between the recessed portion of the seal translating element and each of the coupler fluid path and the connector fluid path.

In accordance with another embodiment of one or more of the inventions presented in this disclosure, an aseptic coupling system includes a coupler and at least one connector. The at least one connector includes a body defining a plug portion and a connector fluid path, and an aseptic cap releasably secured to an end of the plug portion by at least one extension configured to interlock with a corresponding notch on the plug portion. The coupler includes a housing defining at least one socket portion for receiving the plug portion of the at least one connector and a cavity adjacent to the at least one socket portion, a seal translating element assembled with the housing and at least partially disposed within the cavity of the coupler housing, and an actuator element connected to the seal translating element and operable to move the seal translating element from a cap receiving closed position to a cap displacing open position. When the seal translating element is in the cap receiving closed position, the cap of the at least one connector is receivable in the cavity of the coupler housing in alignment with the seal translating element. When the seal translating element is moved from the cap receiving closed position to the cap displacing open position, the at least one extension disengages from the corresponding notch such that the cap is disengaged from the connector plug portion, and a coupler fluid path, defined by the seal translating element, is moved from an offset portion of the coupler housing cavity into alignment with the connector fluid path.

### BRIEF DESCRIPTION OF THE DRAWINGS

Further advantages and benefits will become apparent to those skilled in the art after considering the following description and appended claims in conjunction with the accompanying drawings, in which:

FIG. 1A is a schematic cross-sectional illustration of an exemplary aseptic coupling system including a translating seal mechanism, according to an exemplary embodiment of the present disclosure, shown in an uncoupled condition;

FIG. 1B is a schematic cross-sectional illustration of the aseptic coupling system of FIG. 1A, shown in a coupled condition with the seal translating element in the cap receiving closed position;

FIG. 1C is a schematic cross-sectional illustration of the aseptic coupling system of FIG. 1A, shown in the coupled condition with the seal translating element in the cap displacing open position;

FIG. 2 is a perspective view of an exemplary aseptic coupling system according to an exemplary embodiment of the present disclosure, shown in an uncoupled condition;

FIG. 3 is a cross-sectional view of a capped connector of the aseptic coupling system of FIG. 2;

FIG. 4 is a cross-sectional view of the coupler of the aseptic coupling system of FIG. 2;

FIG. 4A is an exploded perspective view of the coupler of FIG. 4;

FIG. 5 is a cross-sectional view of the aseptic coupling system of FIG. 2, shown in a coupled condition with the plunger in the cap receiving closed position;

FIG. 5A is an enlarged partial cross-sectional view of the connector and coupler sealing portions of the aseptic coupling system of FIG. 2; and

FIG. 6 is a cross-sectional view of the aseptic coupling system of FIG. 2, shown in a coupled condition with the plunger in the cap displacing open position.

### DESCRIPTION OF THE EXEMPLARY EMBODIMENTS

While various inventive aspects, concepts and features of the inventions may be described and illustrated herein as embodied in combination in the exemplary embodiments, these various aspects, concepts and features may be used in many alternative embodiments, either individually or in various combinations and sub-combinations thereof. Unless expressly excluded herein all such combinations and sub-combinations are intended to be within the scope of the present inventions. Still further, while various alternative embodiments as to the various aspects, concepts and features of the inventions—such as alternative materials, structures, configurations, methods, circuits, devices and components, software, hardware, control logic, alternatives as to form, fit and function, and so on—may be described herein, such descriptions are not intended to be a complete or exhaustive list of available alternative embodiments, whether presently known or later developed. Those skilled in the art may readily adopt one or more of the inventive aspects, concepts or features into additional embodiments and uses within the scope of the present inventions even if such embodiments are not expressly disclosed herein. Additionally, even though some features, concepts or aspects of the inventions may be described herein as being a preferred arrangement or method, such description is not intended to suggest that such feature is required or necessary unless expressly so stated. Still further, exemplary or representative values and ranges may be included to assist in understanding the present disclosure, however, such values and ranges are not to be construed in a limiting sense and are intended to be critical values or ranges only if so expressly stated. Parameters identified as “approximate” or “about” a specified value are intended to include both the specified value and values within 10% of the specified value, unless expressly stated otherwise. Further, it is to be understood that the drawings accompanying the present disclosure may, but need not, be to scale, and therefore may be understood as teaching various ratios and proportions evident in the drawings. Moreover, while various aspects, features and concepts may be expressly identified herein as being inventive or forming part of an invention, such identification is not intended to be exclusive, but rather there may be inventive aspects, concepts and features that are fully described herein without being expressly identified as such or as part of a specific invention, the inventions instead being set forth in the appended claims. Descriptions of exemplary methods or processes are not limited to inclusion of all steps as being required in all cases, nor is the order that the steps are presented to be construed as required or necessary unless expressly so stated.

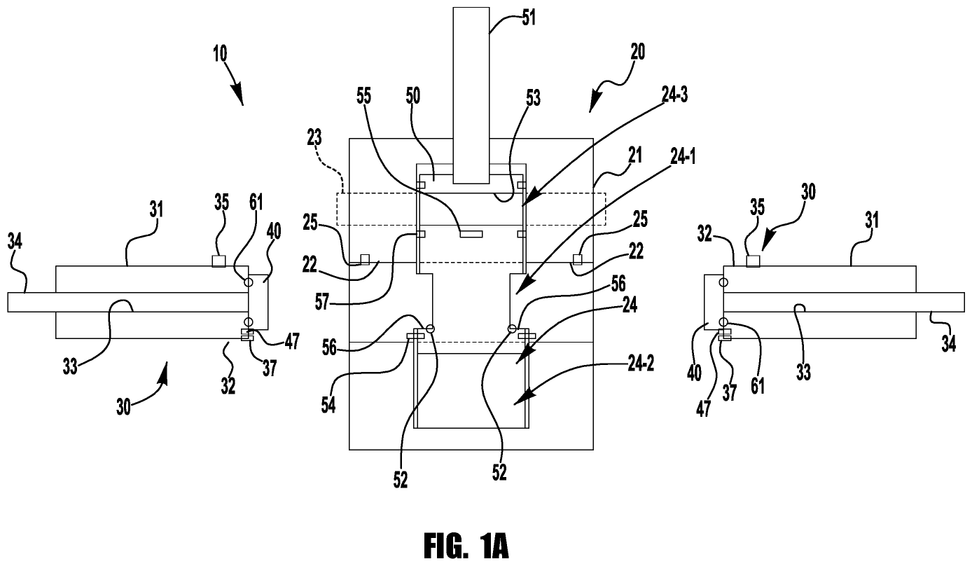

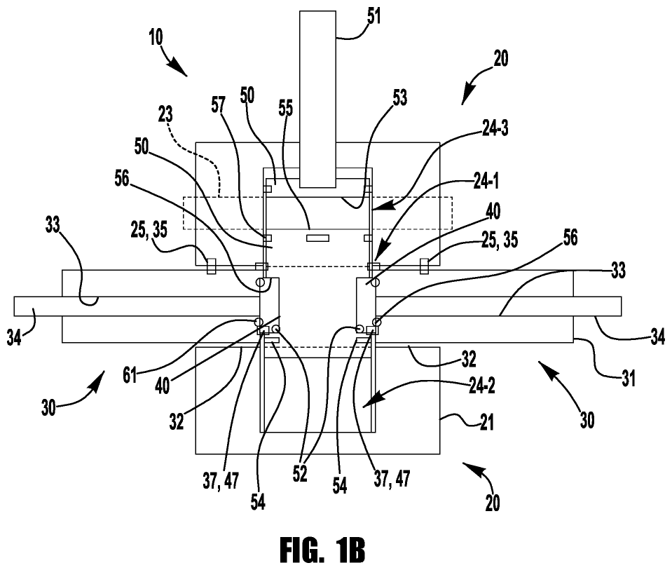

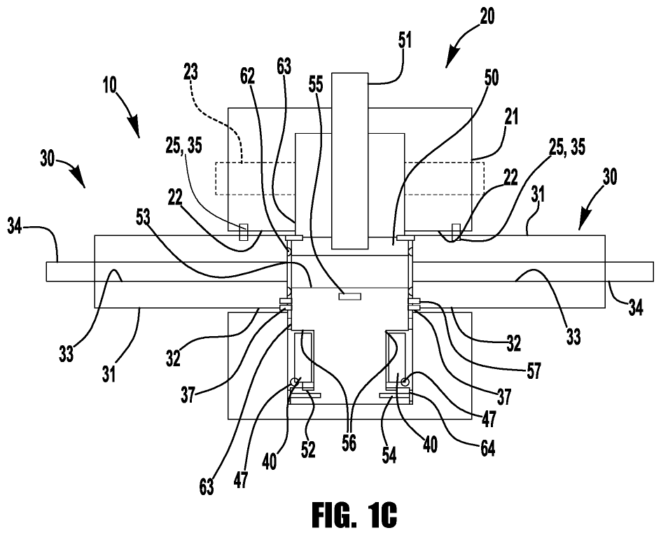

In an exemplary embodiment of the present disclosure, as schematically shown in FIGS. 1A-1C, an aseptic coupling system 10 includes a coupler 20 and at least one connector 30 including a plug portion 32 configured for coupling engagement with a corresponding socket portion 22 of the coupler. While the exemplary illustrated embodiment includes a coupler 20 having two socket portions 22 for coupling with two connectors 30, in other embodiments (not shown), a coupler may include a single socket portion for coupling with a single connector, or three or more socket portions for coupling with a corresponding number of connectors.

The exemplary connectors 30 each include a body 31 defining the plug portion 32 and a connector fluid path 33, and a terminal connection 34 secured to (e.g., integrally formed with or assembled with) the body 31. By providing for mating engagement with the coupler 20, the connectors can be provided as universal genderless elements that do not need to be configured for mating engagement with each other. This may allow, for example, for use of identical connectors for each portion of the fluid system being connected, thereby reducing the number and type of components that are manufactured and stored.

In the exemplary arrangement, each connector 30 includes an aseptic cap 40 releasably secured to an end of the plug portion 32 by an attachment feature 47 (e.g., detent, extension, tab, latch, press fit, interference fit, magnetic coupling, multi-use adhesive attachment, etc.) on the cap that releasably engages a corresponding attachment feature 37 on the plug portion. A sealing element 61 (e.g., O-ring, gasket, plastic/elastomeric overmold) may be provided between the plug portion 32 and the cap 40 to provide a seal (e.g., fluid tight seal, particulate impeding seal) between the cap and the connector fluid path 33, for example, to prevent contamination of the fluid path.

The connector body 31 may be provided with a coupler attachment feature 35 (e.g., detent, extension, tab, latch, press fit, interference fit, magnetic coupling, multi-use adhesive attachment, etc.) for releasable attachment of the connector 30 with a corresponding attachment feature 25 of the coupler 20 upon full engagement (e.g., a fully inserted position) of the connector plug portion 32 with the coupler socket portion 22.

The exemplary coupler 20 includes a housing 21 defining the socket portion(s) 22, a cavity 24 adjacent to the socket portion(s) (e.g., between the socket portions), and a seal translating element 50 assembled with the housing 21 and at least partially disposed within the cavity of the coupler housing. An actuator element 51 is connected or fixed to (e.g., integrally formed with or assembled with) the seal translating element 50, and is operable to move the seal translating element between a cap receiving closed position (FIG. 1B) and a cap displacing open position (FIG. 1C). In the cap receiving closed position, recessed portions 56 of the seal translating element 50 are positioned (e.g., in alignment with the socket portions 22) to receive the caps 40 of the connectors 30, in an inline portion 24-1 of the coupler housing cavity 24, when the connector plug portions 32 are fully inserted into the coupler socket portions 22. When the seal translating element 50 is moved from the cap receiving closed position to the cap displacing open position, the caps 40 are disengaged from the connector plug portions 32 and displaced to a first offset portion 24-2 of the coupler housing cavity 24, and a coupler fluid path 53, defined by the seal translating element 50, is moved from a second offset portion 24-3 of the coupler housing cavity into alignment with the connector fluid paths 33 to permit fluid flow between and through the connectors 30.

Additionally, according to another aspect of the present disclosure, the seal translating element 50 may be provided with an interlocking feature 57 (e.g., detent, tab, latch, press fit, interference fit, etc.), as schematically shown in FIGS. 1A-1C, that interlocks with the connector plug interlocking feature 37 when the plunger frame is in the cap displacing open position, for example, to impede intentional or inadvertent detachment of the connector(s) from the coupler in the open position.

The coupling system may use a variety of sealing arrangements to provide a leak tight seal around the open fluid path 53, 33 when the seal translating element 50 is in the cap displacing open position, including, for example, annular seal portions 62 (e.g., O-rings, gaskets, overmolded plastic/elastomeric portions) surrounding and disposed between the coupler fluid path 53 and the connector fluid paths 33 (FIG. 1C). Additionally, sealing arrangements may be provided between the recessed cap retaining portions 56 of the seal translating element 50 and each of the coupler fluid path 53 and the connector fluid paths 33, for example, to isolate the exterior surfaces of the caps 40 (which may be exposed to contaminants) from the system fluid path 53, 33. For example, annular sealing portions (e.g., O-rings, gaskets, overmolded plastic/elastomeric portions) may be provided between the connector plug portions 32 and the seal translating element 50 (at 63) and between the coupler housing 21 and the seal translating element 50 (at 64).

In an exemplary use of the system 10 of FIGS. 1A-1C, to produce an aseptic connection between first and second connectors 30, the plug portions 32 of the connectors are inserted into the socket portions 22 of the coupler 20, such that the connector caps 40 are received in the recessed cap retaining portions 56 of the seal translating element 50, and coupler interlocking features 35 of the connector bodies 31 engage the corresponding interlocking features 25 of the coupler 20 to secure the connectors 30 to the coupler 20. In some uses of the system 10, at least one of the connectors 30 may be preinstalled with the coupler 20 (e.g., by the manufacturer or supplier), with the other(s) of the connectors being installed with the coupler by the end user.

In this coupled condition, the actuator element 51 is operated to move the seal translating element 50 from the cap receiving closed position to the cap displacing open position. By this actuation, the coupler fluid path 53 is moved from the second offset portion 24-3 of the coupler housing cavity 24 to the inline portion 24-1 of the coupler housing cavity, into alignment with the connector fluid paths 33. The received caps 40 detach from the corresponding connector plug portions 32 and are moved to the first offset portion 24-2 of the coupler housing cavity 24, where the caps are sealingly enclosed and isolated from the system fluid path 53, 33.

To disconnect the connectors 30, the actuator element 51 is operated to return the seal translating element 50 to the cap receiving closed position, thereby moving the coupler fluid path 53 to the second offset portion 24-3, and the recessed cap retaining portions 56, with retained caps 40, to the inline portion 24-1, thereby re-engaging the caps with the connector plug portions 32. The capped connectors 30 may then be withdrawn from the coupler 20.

In other embodiments, an aseptic coupling system may be designed for single use. In such an arrangement, rather than retaining the caps in recessed portions of the seal translating element, the seal translating element may instead merely detach the caps from the plug portions, and eject the caps from the coupler, or into a cavity within the coupler housing. In such an arrangement, the connectors may (but need not) be configured to more permanently attach to the coupler housing, for example, to prevent an attempt to re-use the connectors or the coupler.

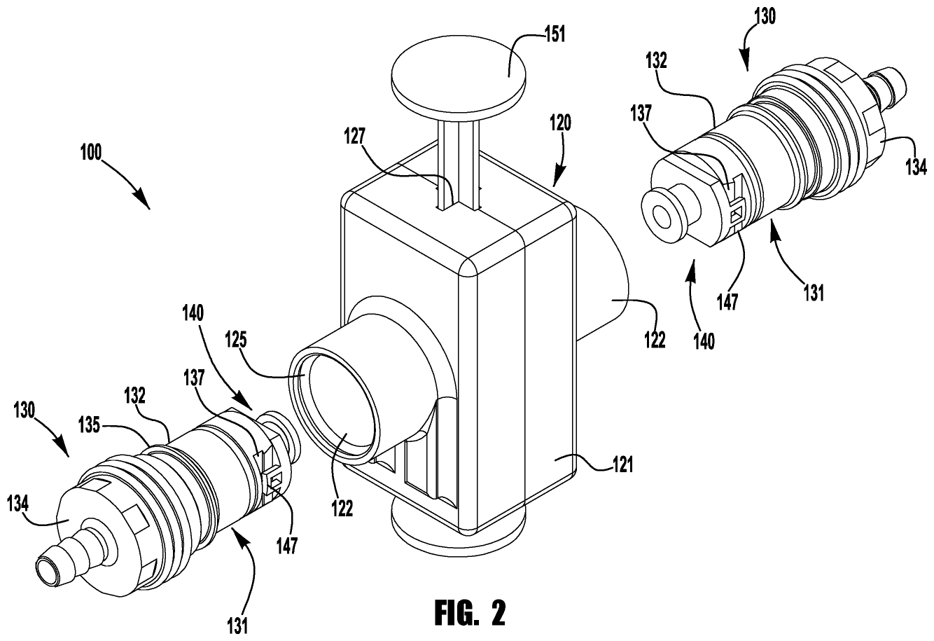

FIGS. 2-6 illustrate an exemplary embodiment of an aseptic coupling system 100 including a variety of features, any one or more of which may be incorporated into the more broadly described exemplary aseptic coupling system of FIGS. 1A-1C.

The exemplary aseptic coupling system 100 includes a coupler 120 and at least one connector 130 including a plug portion 132 configured for coupling engagement with a corresponding socket portion 122 of the coupler. While the exemplary illustrated embodiment includes a coupler 120 having two socket portions 122 for coupling with two connectors 130, in other embodiments (not shown), a coupler may include a single socket portion for coupling with a single connector (e.g., with a second end connection or terminal connection integrally formed with the coupler), or three or more socket portions for coupling with a corresponding number of connectors. Additionally, while the socket portions 122 of the exemplary coupler 120 are coaxially aligned, in other embodiments (not shown), two or more socket portions may be axially offset (e.g., parallel), perpendicular to each other, or oriented at different angles with respect to each other, for example, to form elbow, T-shaped, Y-shaped, or multi-ported manifold-type configurations.

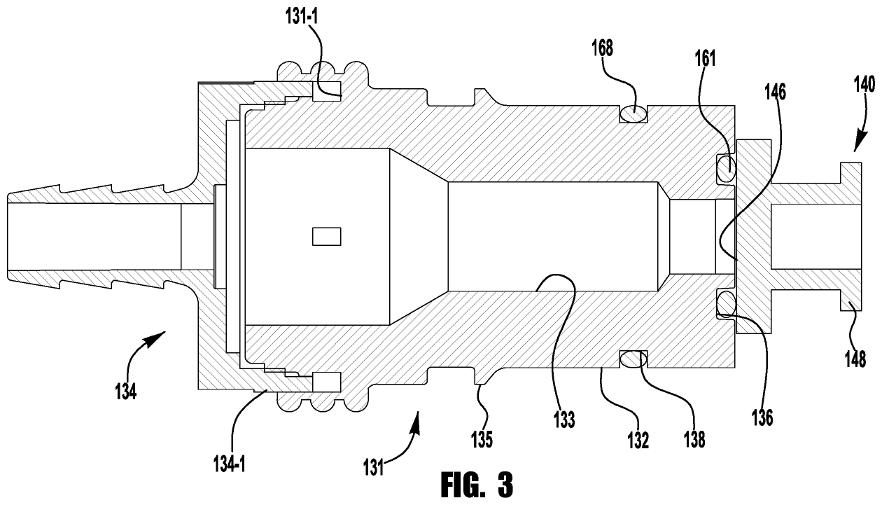

The exemplary connectors 130 each include a body 131 defining the plug portion 132 and a connector fluid path 133 (FIG. 3), and a terminal connection 134 assembled with the body 131. While many different types of attachment may be used, in the illustrated example, a collar portion 134-1 (FIG. 3) of the terminal connection 134 is press fit into an annular recess 131-1 of the connector body 131. The illustrated terminal connections 134 include barbed hose fittings for installation with plastic hose ends. In other embodiments, other types of terminal connections may be used on one or more of the connectors, including, for example, tube fittings, pipe fittings, zero clearance fittings, and weld ends.

Each connector 130 includes an aseptic cap 140 releasably secured to an end of the plug portion 132. Many different types of caps may be used. In the illustrated embodiment, the caps include a solid inner end face 146 for sealing off the connector fluid path 133, and an outer flange portion or other such extension 148 (which may, but need not, be hollow as shown). This flange portion 148 may facilitate alignment of the caps during installation of the connectors. In other embodiments, the caps may be provided without any extension structure (e.g., instead having a disc shaped plug covering portion).

The exemplary caps 140 are releasably secured to the plug portions 132 by an interlocking feature 147 on the cap that releasably engages a corresponding interlocking feature 137 on the plug portion. While many different suitable interlocking arrangements may be utilized, in the illustrated embodiment, the caps may be provided with flange-barb ended tab portions or other such extensions 147 that snap or slide into interlocking engagement with notched or grooved portions 137 in the ends of the connector plug portions 132, thereby securing the cap 140 against axial separation from the connector plug portion 132 while permitting actuator operated translational detachment of the cap from the plug portion, as described in greater detail below. A flexed, interference fit between the tab portions 147 and groove portions 137 may be provided to facilitate retention of the cap 140 on the connector plug portion 132.

A sealing element 161 (FIG. 3) is provided between the plug portion 132 and the cap 140 to provide a seal (e.g., fluid tight seal, particulate impeding seal) between the cap and the connector fluid path 133, for example, to prevent contamination of the fluid path. In the illustrated example, O-ring (or other gasket) seals 161 are disposed in annular end face grooves 136 in the connector plug portions 132 for sealing engagement with corresponding end face surfaces 146 of the caps 140 prior to connection with the coupler 120. The plug portion 132 may additionally be provided with an O-ring or gasket seal 168 retained in an outer circumferential groove 138, for example, for sealing engagement with the corresponding coupler socket portion 122. In other embodiments (not shown), O-ring (or other gasket) seals may be disposed in end face grooves in the caps. Still other sealing arrangements may be used, including, for example, overmolded elastomeric/plastic portions or plastic-on-plastic interference fit geometries. The sealing elements may be provided in a variety of suitable materials, including, for example, any one or more of silicone, ethylene propylene diene monomer (EPDM), fluoroelastomers (e.g., Viton™), Buna-N, Natural Rubber, Polyisoprene, FFKM, FKM, FVMQ (fluorosilicone), Nitrile, thermoplastic polyurethane (TPU), thermoplastic vulcanisates (TPV), and thermoplastic elastomers (TPE).

The connector body 131 is provided with a coupler interlocking feature 135 for releasable interlocking attachment of the connector 130 with a corresponding interlocking feature 125 of the coupler 120 upon full engagement (e.g., a fully inserted position) of the connector plug portion 132 with the coupler socket portion 122. In the illustrated example, the connector body 131 is provided with an annular rib 135 that snaps into engagement with an inner peripheral groove 125 in the coupler socket portion 122 when the connector plug portion 132 is fully inserted into the coupler socket portion. Other interlocking features may additionally or alternatively be used, including, for example, press fit engagement, bayonet-style connections, snap and/or hook and loop type fasteners, screw arrangements, twist-to-lock arrangements, ball detents, button depressible latch arrangements, and slide lock arrangements.

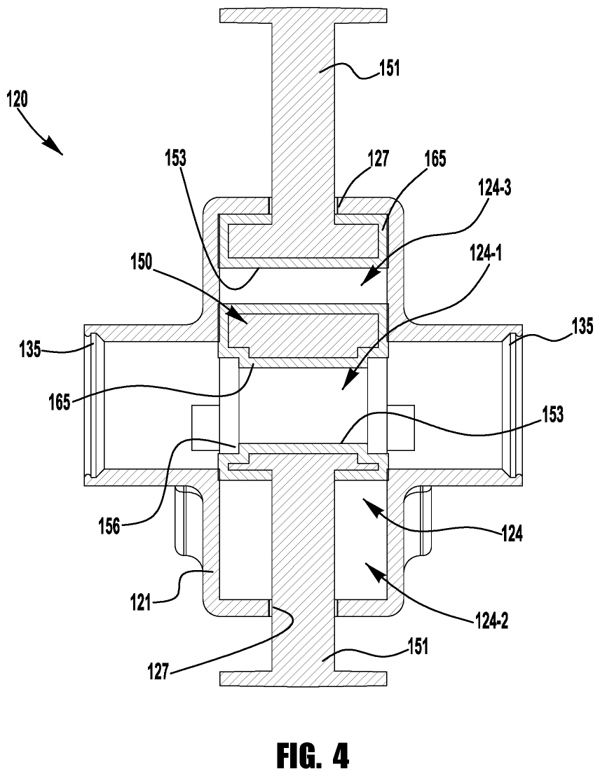

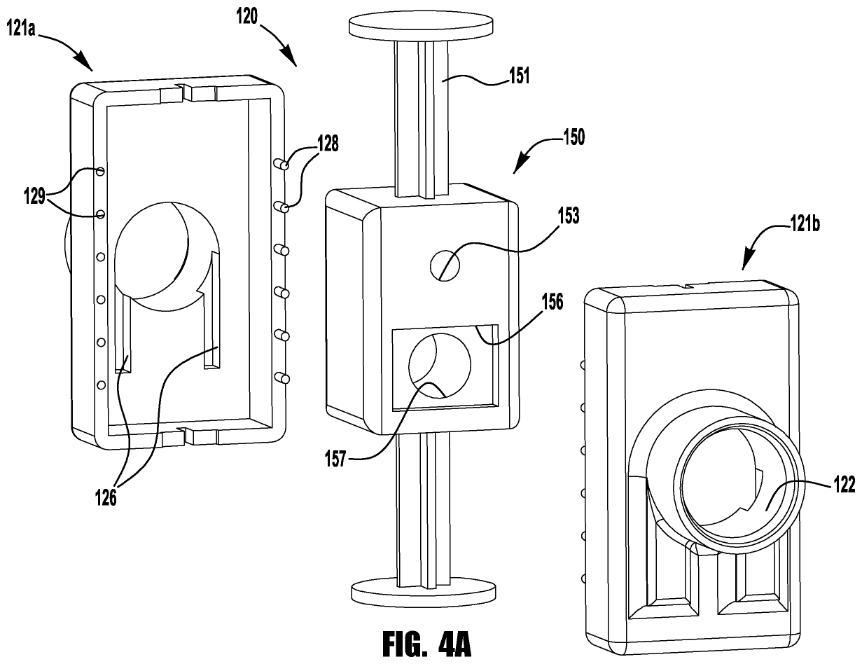

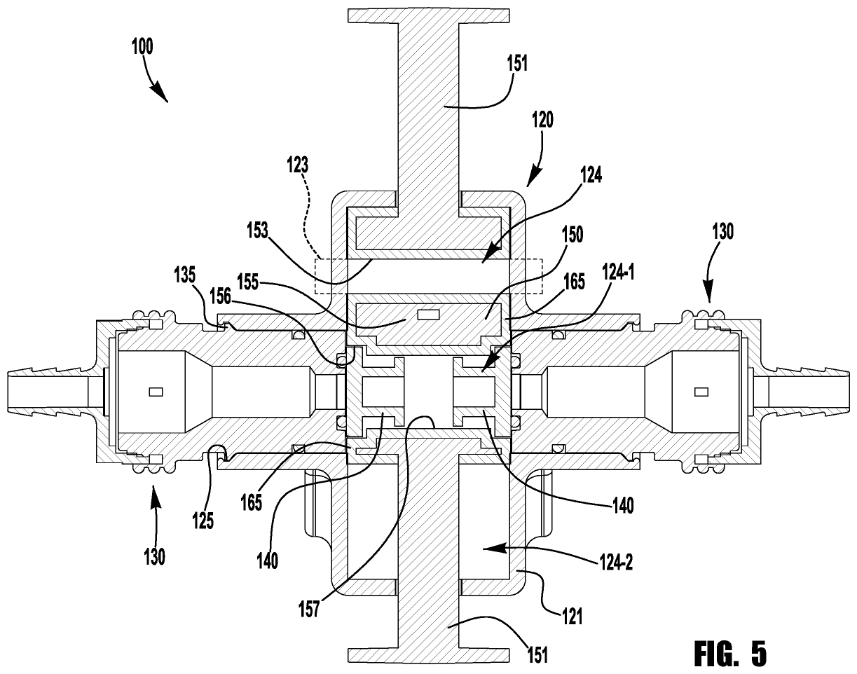

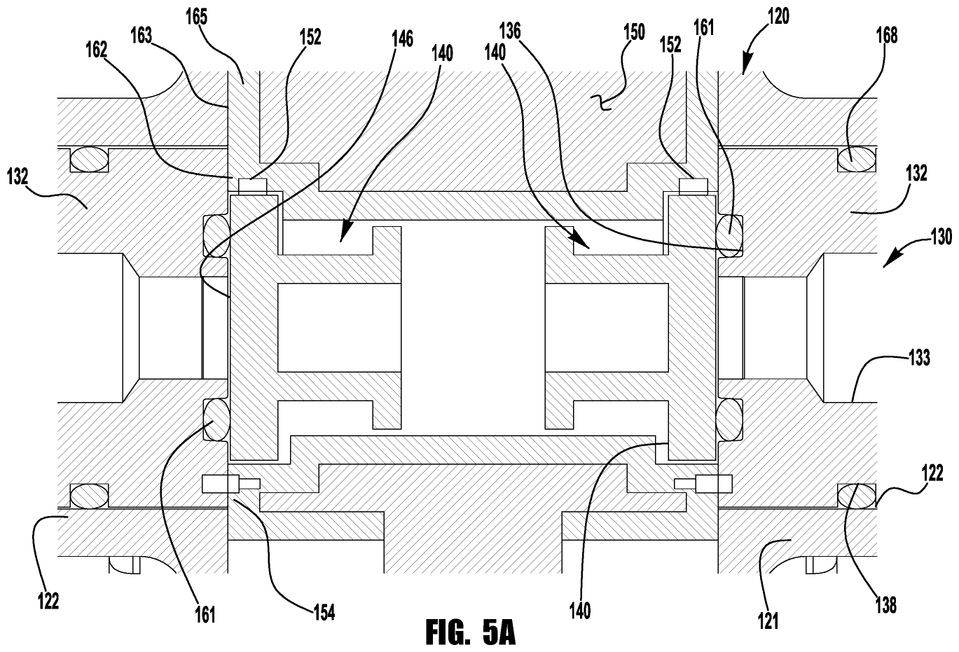

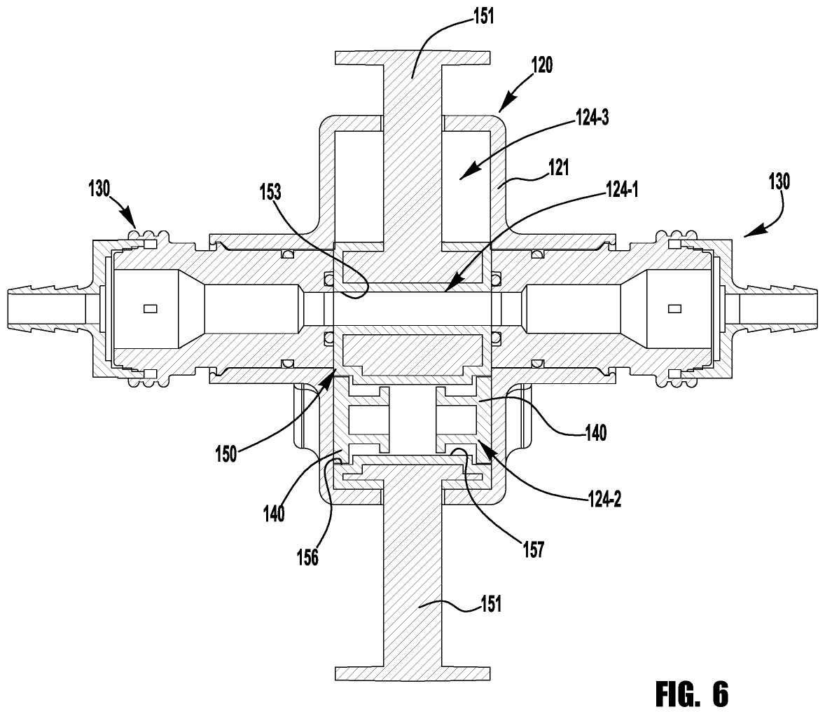

The exemplary coupler 120 includes a housing 121 defining the socket portions 122 and a cavity 124 (FIG. 4) disposed between the socket portions, and a seal translating plunger frame 150 assembled with the housing 121 and at least partially disposed within the cavity of the coupler housing. A plunger actuator 151 is connected to (e.g., integrally formed with, as shown) the plunger frame 150, and extending through openings 127 in the ends of the coupler housing 121. The plunger actuator 151 is operable (e.g., by pushing or pulling movement) to laterally slide the plunger frame between a cap receiving closed position (FIG. 5) and a cap displacing open position (FIG. 6). In the cap receiving closed position, recessed portions 156 and a through bore 157 of the seal translating plunger frame 150 are positioned (e.g., in alignment with the socket portions 122) to receive the caps 140 of the connectors 130, in an inline portion 124-1 of the coupler housing cavity 124, when the connector plug portions 132 are fully inserted into the coupler socket portions 122.

When the seal translating plunger frame 150 is moved from the cap receiving closed position to the cap displacing open position (e.g., by pushing or pulling movement of the plunger actuator 151), the caps 140 are laterally (i.e., perpendicular to the central axis of the fluid flow path) disengaged from the connector plug portions 132 and laterally displaced to a first offset portion 124-2 of the coupler housing cavity 124, and a coupler fluid path 153 in the plunger frame 150 is laterally moved from a second offset portion 124-3 of the coupler housing cavity into alignment with the connector fluid paths 133 to permit fluid flow between and through the connectors 130. In the illustrated example, the caps 140 are displaced in the direction of the connector plug grooved portions 137, perpendicular to the fluid path axis, such that the cap tabs 147 slide out of engagement with the grooved portions. To ensure alignment of the grooved portions 137 with the sliding direction of the plunger frame 150, the connectors 130 and coupler 120 may have a keyed arrangement to limit plug insertion to this orientation. In the illustrated embodiment, the cap portions 140 include a flatted portion 149 that corresponds to a flatted recess 159 in the plunger frame to limit installation of the connector 130 to this aligned orientation. Other shaped keyed arrangements may additionally or alternatively be used. In other embodiments (not shown), the keyed arrangement may be provided on the connector body (e.g., the plug portion) and the coupler housing (e.g., the socket portion). These keyed arrangements, or additional keyed arrangements, may also be effective to prevent twisting rotation of the installed connector within the coupler.

To accommodate the interlocking tabs 147, the coupler housing 121 includes grooves 126 (see FIG. 4A) recessed from the cavity 124 and extending downward from the socket portions 122, which allow the connector plug portions 132 to be fully retained within the couple socket portions without extending into the cavity, and prevent rotation or misalignment of the caps 140 when disengaged from the connector plug portions.

Additionally, according to another aspect of the present disclosure, the plunger frame may be provided with an interlocking feature (e.g., tabbed portion, not shown) that interlocks with the connector plug notched portions when the plunger frame is in the cap displacing open position, for example, to impede intentional or inadvertent detachment of the connector(s) from the coupler in the open position.

Other types of seal translating elements and actuating elements may additionally or alternatively be used. As contemplated by the present disclosure, a coupling system may include any suitable element capable of displacing the caps out of alignment with the connector plug portions and moving the coupler fluid path into alignment with the plug portions. For example, the seal translating element may include a rotatable component, a pivoting component, a screw threaded component, or an axially slideable component, and the actuating element may include a corresponding user operable actuating component, such as, for example, a handle/wheel, lever, or toggle switch. In still other embodiments, the actuating element may be configured for remote actuation, utilizing, for example, electronic, electromagnetic, hydraulic, and/or pneumatic actuation mechanisms.

Referring back to the exemplary embodiment of FIGS. 2-6, the O-ring seals 161 provide for a seal around and between the coupler fluid path 153 and the connector fluid paths 133 when the plunger frame 150 is in the cap displacing open position. The plunger frame 150 is provided with an overmolded elastomeric (e.g., silicone, ethylene EPDM, fluoroelastomers, Buna-N, Natural Rubber, Polyisoprene, FFKM, FKM, FVMQ, Nitrile, TPU, TPV, and TPE) seal layer 165 that defines sealing portions between the connector plug portions 132 and the seal translating element 150 (at 162) and between the coupler housing 121 and the seal translating element 150 (at 163). These sealing portions 162, 163 effect a seal (e.g., fluid tight seal, particulate impeding seal) between the recessed cap retaining portions 156 of the seal translating element 150 and each of the coupler fluid path 153 and the connector fluid paths 133, for example, to isolate the exterior surfaces of the caps 140 (which may be exposed to contaminants) from the system fluid path 153, 133. A variety of overmolding seal arrangements may be used, including, for example, any one or more of the above listed elastomers provided at a thickness of about 0.020″ to about 0.120″ to provide sufficient elasticity. As shown, the overmolding may extend over the surfaces of the plunger frame 150, terminating at the actuating plungers 151. In an exemplary embodiment, overmolded surfaces of the coupler fluid path 153 may facilitate maintenance of an aseptic flow path (e.g., by chemical compatibility and/or eliminating seams or crevices in the flow path). In other embodiments, the overmolding may be further limited to areas of the plunger frame at or immediately surrounding the sealing portions, or may extend further to cover the actuating plungers.

In still other embodiments, other types of sealing portions (e.g., O-rings, gaskets, plastic elements) may be provided between the connector plug portions 132 and the plunger frame 150 (or other such seal translating element) and between the coupler housing 121 and the plunger frame.

In an exemplary use of the system 10 of FIGS. 2-6, to produce an aseptic connection between first and second connectors 130, the plug portions 132 of the connectors are inserted into the socket portions 122 of the coupler 120, such that the connector caps 140 are received in the recessed cap retaining portions 156 of the plunger frame 150, and the annular ribs 135 of the connector bodies 131 snap into engagement with the inner peripheral grooves 125 of the coupler socket portions 122 to secure the connectors 130 to the coupler 120. In some uses of the system 100, at least one of the connectors 130 may be preinstalled with the coupler 120 (e.g., by the manufacturer or supplier), with the other(s) of the connectors being installed with the coupler by the end user.

In this coupled condition, the plunger actuator 151 is operated (pushed or pulled) to slide the plunger frame 150 from the cap receiving closed position to the cap displacing open position. By this actuation, the coupler fluid path 153 is moved from the second offset portion 124-3 of the coupler housing cavity 124 to the inline portion 24-1 of the coupler housing cavity, into alignment with the connector fluid paths 133. The received caps 140 detach from the corresponding connector plug portions 132 and are moved to the offset portion 124-2 of the coupler housing cavity 124, where the caps are sealingly enclosed and isolated from the system fluid path 153, 133.

To disconnect the connectors 130, the plunger actuator 151 is operated to return the plunger frame 151 to the cap receiving closed position, thereby moving the coupler fluid path 153 to the second offset portion 124-3, and the recessed cap retaining portions 156, with retained caps 140, to the inline portion 124-1, thereby re-engaging the caps with the connector plug portions 132. The capped connectors 130 may then be withdrawn from the coupler 120.

In other embodiments, an aseptic coupling system may be designed for single use. In such an arrangement, rather than retaining the caps in recessed portions of the seal translating element, the seal translating element may instead merely detach the caps from the plug portions, and eject the caps from the coupler, or into a cavity within the coupler housing. In such an arrangement, the connectors may (but need not) be configured to more permanently attach to the coupler housing, for example, to prevent an attempt to re-use the connectors or the coupler.

In accordance with other aspects of the present disclosure, other features may additionally or alternatively be provided with the exemplary aseptic coupling systems described herein. As one example, the seal translating element 50 or plunger frame 150 may be provided with a latching or interlocking feature (shown schematically at 52 in FIGS. 1A-1C and at 152 in FIG. 5A) to more securely hold the caps 40, 140 with the seal translating element 50, 150 upon full insertion of the connector plug portions 32, 132 into the coupler socket portions 22, 122.

As another example, the coupler housing 21, 121 may be provided with at least one septum, plugged connection, or other such access port (shown schematically at 23 in FIGS. 1A-1C and at 123 in FIG. 5), aligned with the second offset portion 24-3, 124-3 of the coupler housing cavity 124. When the seal translating element/plunger frame 50, 150 is in the cap displacing open position, the access port 23, 123 aligns with the coupler fluid path 53, 153, for example, to purge or extract any fluid trapped in the coupler fluid path.

As another example, the seal translating element/plunger frame 50, 150 may be provided with a latching element (shown schematically at 54 in FIGS. 1A-1C and at 154 in FIG. 5A) that blocks or impedes movement of the seal translating element/plunger frame 50, 150 from the closed position to the open position unless and until the connector plug portions 32, 132 are fully inserted into the coupler socket portions 22, 122. In one such example, spring loaded latches 54, 154 may be depressed by the fully inserted plug portions 32, 132 to provide clearance for lateral sliding movement of the seal translating element/plunger frame 50, 150.

As another example, the coupling system may be provided with one or more sensors (e.g., at or near the coupler fluid path 53, 153, as shown schematically at 55 in FIGS. 1A-1C and at 155 in FIG. 5) configured to measure and transmit (e.g., by wireless Bluetooth® or WiFi communication), fluid flow data, including, for example, flow rate, temperature, and/or pressure.

While any suitable materials may be used, in exemplary embodiments, the plug bodies 31, 131, terminal connections 34, 134, coupler housing 21, 121, and seal translating element/plunger frame 50, 150, and actuating element 51, 151 may include any one or more of polypropylene, nylon, polyethylene, acetal, polycarbonate, polysulfone, Acrylic, PBT, ABS, and/or copolyester. The aseptic caps 40, 140 may include rigid plastics and/or elastomeric materials, such as, for example, the exemplary materials described herein. As shown in the exploded view of FIG. 4A, the coupler housing may be formed from injection molded housing halves 121a, 121b, for example, to facilitate formation of internal features of the housing, with the housing halves including pins 128 and sockets 129 for press fit assembly of the housing halves around the plunger frame 150. As shown, the housing halves 121a, 121b may, but need not, be identical components, for example, to minimize manufacturing and inventory costs.

The inventive aspects have been described with reference to the exemplary embodiments. Modification and alterations will occur to others upon a reading and understanding of this specification. It is intended to include all such modifications and alterations insofar as they come within the scope of the appended claims or the equivalents thereof.

1. An aseptic coupling system comprising:
first and second connectors, each comprising:
a body defining a plug portion and a connector fluid path; and
an aseptic cap releasably secured to an end of the plug portion by an attachment feature on the cap that releasably engages the plug portion for attachment thereto; and
a coupler comprising:
a housing defining first and second socket portions for receiving the plug portions of the first and second connectors, and a cavity disposed between the first and second socket portions;
a seal translating element assembled with the housing and at least partially disposed within the cavity of the coupler housing; and
an actuator element connected to the seal translating element and operable to move the seal translating element from a cap receiving closed position to a cap displacing open position;
wherein when the seal translating element is in the cap receiving closed position, the caps of the first and second connectors are receivable in the cavity of the coupler housing in alignment with the seal translating element; and
when the seal translating element is moved from the cap receiving closed position to the cap displacing open position, the caps are disengaged from the connector plug portions and a coupler fluid path, defined by the seal translating element, is moved from an offset portion of the coupler housing cavity into alignment with the connector fluid paths.

2. The aseptic coupling system of claim 1, wherein when the seal translating element is in the cap receiving closed position, first and second recessed portions of the seal translating element are positioned to receive the caps of the first and second connectors.

3. The aseptic coupling system of claim 2, wherein the coupler further comprises sealing arrangements between the first and second recessed portions of the seal translating element and each of the coupler fluid path and the connector fluid paths.

4. The aseptic coupling system of claim 2, wherein the first and second recessed portions of the seal translating element are configured to retain the caps when the seal translating element is moved from the cap receiving closed position to the cap displacing open position.

5. The aseptic coupling system of claim 4, wherein the actuator element is operable to move the seal translating element from the cap displacing open position back to the cap receiving closed position.

6. The aseptic coupling system of claim 5, wherein when the seal translating element is moved from the cap displacing open position back to the cap receiving closed position, the caps are re-attached to the plug portions of the first and second connectors.

7. (canceled)

7. The aseptic coupling system of claim 1, wherein the attachment feature on each cap comprises at least one extension configured to interlock with a corresponding notch on the plug portion, the at least one extension disengaging from the corresponding notch when the seal translating element is moved from the cap receiving closed position to the cap displacing open position.

8. The aseptic coupling system of claim 7, wherein the housing includes a recessed groove that receives the at least one extension when the seal translating element is moved from the cap receiving closed position to the cap displacing open position.

9. (canceled)

10. The aseptic coupling system of claim 1, wherein the housing includes an access port that aligns with the coupler fluid path when the seal translating element is in the cap displacing open position.

11. The aseptic coupling system of claim 10, wherein the access port comprises at least one of a septum and a plugged connection.

12. (canceled)

14. An aseptic coupling system comprising:
at least one connector, each comprising a body defining a plug portion and a connector fluid path and an aseptic cap releasably secured to an end of the plug portion by an attachment feature on the cap that releasably engages the plug portion for attachment thereto; and
a coupler comprising a housing defining at least one socket portion for receiving the plug portion of the at least one connector and a cavity adjacent to the at least one socket portion, a seal translating element assembled with the housing and at least partially disposed within the cavity of the coupler housing, and an actuator element connected to the seal translating element and operable to move the seal translating element between a cap receiving closed position and a cap displacing open position;
wherein when the seal translating element is in the cap receiving closed position, a recessed portion of the seal translating element is positioned to receive the cap of the at least one connector; and
when the seal translating element is moved from the cap receiving closed position to the cap displacing open position, the cap is disengaged from the connector plug portion and displaced to a first offset portion of the coupler housing cavity, and a coupler fluid path, defined by the seal translating element, is moved from a second offset portion of the coupler housing cavity into alignment with the connector fluid path; and
wherein the coupler further comprises sealing arrangements between the recessed portion of the seal translating element and each of the coupler fluid path and the connector fluid path.

15. The aseptic coupling system of claim 14, wherein the recessed portion of the seal translating element is configured to retain the cap when the seal translating element is moved from the cap receiving closed position to the cap displacing open position.

16. The aseptic coupling system of claim 15, wherein the actuator element is operable to move the seal translating element from the cap displacing open position back to the cap receiving closed position.

17. The aseptic coupling system of claim 16, wherein when the seal translating element is moved from the cap displacing open position back to the cap receiving closed position, the cap is re-attached to the plug portion of the at least one connector.

18-21. (canceled)

22. The aseptic coupling system of claim 14, wherein the housing includes an access port that aligns with the coupler fluid path when the seal translating element is in the cap displacing open position.

23. The aseptic coupling system of claim 22, wherein the access port comprises at least one of a septum and a plugged connection.

24-26. (canceled)

27. An aseptic coupling system comprising:
at least one connector, each comprising a body defining a plug portion and a connector fluid path and an aseptic cap releasably secured to an end of the plug portion by at least one extension configured to interlock with a corresponding notch on the plug portion; and
a coupler comprising a housing defining at least one socket portion for receiving the plug portion of the at least one connector and a cavity adjacent to the at least one socket portion, a seal translating element assembled with the housing and at least partially disposed within the cavity of the coupler housing, and an actuator element connected to the seal translating element and operable to move the seal translating element from a cap receiving closed position to a cap displacing open position;
wherein when the seal translating element is in the cap receiving closed position, the cap of the at least one connector is receivable in the cavity of the coupler housing in alignment with the seal translating element; and
when the seal translating element is moved from the cap receiving closed position to the cap displacing open position, the at least one extension disengages from the corresponding notch such that the cap is disengaged from the connector plug portion, and a coupler fluid path, defined by the seal translating element, is moved from an offset portion of the coupler housing cavity into alignment with the connector fluid path.

28-29. (canceled)

30. The aseptic coupling system of claim 27, wherein the actuator element is operable to move the seal translating element from the cap displacing open position back to the cap receiving closed position.

31. The aseptic coupling system of claim 30, wherein when the seal translating element is moved from the cap displacing open position back to the cap receiving closed position, the cap is re-attached to the plug portion of the at least one connector.

32. (canceled)

33. The aseptic coupling system of claim 27, wherein the housing includes a recessed groove that receives the at least one extension when the seal translating element is moved from the cap receiving closed position to the cap displacing open position.

34-39. (canceled)
